(19) United States
(12) Patent Application Publication
(10) Pub. No.: US 2010/0280355 A1
(43) Pub. Date: Nov. 4, 2010

(54) SYSTEM AND METHOD TO CHARACTERIZE CARDIAC FUNCTION

(76) Inventors: Richard A. Grimm, Chagrin Falls, OH (US); Pascal Lim, Paris (FR)

Correspondence Address: TAROLLI, SUNDHEIM, COVELL & TUMMINO L.L.P., 1300 EAST NINTH STREET, SUITE 1700, CLEVELAND, OH, 44114, US

(21) Appl. No.: 12/808,140

(22) PCT Filed: Dec. 11, 2008
(86) PCT No.: PCT/US08/86475

§ 371 Date: Jun. 14, 2010

Related U.S. Application Data
(60) Provisional application No. 61/013,880, filed on Dec. 14, 2007.

(51) Int. Cl. A61B 5/055; A61B 5/02; A61B 8/00

(52) U.S. Cl. 600/411; 600/508; 600/437

(57) ABSTRACT

Systems and methods can quantify cardiac function. In one embodiment, a method (10) for quantifying cardiac function for a patient's heart includes determining (12) an end-systolic strain for each of a plurality of myocardial segments at end systole and determining (14) a peak strain in each of the plurality of myocardial segments. A difference between the peak strain and the end-systolic strain is computed (16) for each of the plurality of myocardial segments. A strain delay index is computed (18) from the computed differences.

### RELATED APPLICATION

This application claims the benefit of U.S. Provisional Patent Application No. 61/013,880, which was filed on Dec. 14, 2007, entitled SYSTEM AND METHOD FOR CHARACTERIZING MYOCARDIAL DYSSYNCHRONY, the entire contents of which is incorporated herein by reference.

### GOVERNMENT INTEREST

This work was supported in part by the National Space Biomedical Research Institute through NASA NCC 9-58, the Department of Defense (Ft. Dietrich, Md., USAMRMC) through Grant #02360007. This work is also supported in part by the National Institutes of Health, National Center for Research Resources, General Clinical Research Center through Grant MO1 RR-018390. The U.S. Government has certain rights in the invention.

### TECHNICAL FIELD

The invention relates to health and, more particularly, to system and method to characterize cardiac function.

### BACKGROUND

Several clinical trials have confirmed the sustained benefit of Cardiac Resynchronization Therapy (CRT) in patients with symptomatic severe left ventricular (LV) dysfunction and wide QRS duration. The beneficial effects of CRT include improvement of symptoms, ejection fraction (EF), mitral regurgitation, LV remodeling, and survival. Despite these encouraging results, a large percentage of patients selected according to QRS duration criteria may not respond to CRT. Observational studies have consistently demonstrated that the main predictor of responsiveness to CRT is mechanical rather than electrical dyssynchrony. Measurement of regional longitudinal myocardial electrical-mechanical events using velocity data acquired with tissue Doppler imaging (TDI) has been shown to enhance the identification of mechanical dyssynchrony and hence, patient selection for those likely to respond to CRT. However limitations of this technique exist, including the lack of specificity related to delayed longitudinal contraction in patients with an ischemic cardiomyopathy.

Patients with significant mechanical dyssynchrony may be non-responsive because desynchronized segments may be scarred and therefore lack a certain degree of residual contractility. This phenomenon is particularly evident for ischemic patients who have myocardial segments with delayed contraction, such as may result from scar as opposed non-ischemic and primary conduction myopathies. Existing identification of responders simply by time delay indices seems inherently limited. Accordingly, an improved approach to quantify cardiac function which can be utilized to predict response to CRT is desired.

### SUMMARY

The invention relates to a system and method to characterize cardiac function. For instance, a method can be employed to compute a quantity, strain delay index, which represents a summation of the difference between peak contractility and end-systolic contractility across a set of myocardial segments. The method can be implemented as computer executable instructions programmed to compute the strain delay index based on image data (e.g., ultrasound image data utilizing speckle tracking) acquired for a patient's heart or based on another mechanism that quantifies wall motion.

One embodiment of the invention relates to a method for quantifying cardiac function and which may also be employed to predict a response to CRT. The method includes determining an end-systolic strain for each of a plurality of myocardial segments at end systole and determining a peak strain for each of the plurality of myocardial segments. A difference between the peak strain and the end-systolic strain is computed for each of the plurality of myocardial segments. A strain delay index is computed from the differences computed for the plurality of myocardial segments.

Another aspect of the invention relates to a method for quantifying cardiac function for a patient's heart. The method can include computing a summation of a difference between peak contractility and end-systolic contractility across a plurality of myocardial segments of a chamber of the patient's heart to provide a strain delay index, whereby a response to cardiac resynchronization therapy is predictable according to a value of the strain delay index.

Still another aspect of the invention provides a system for quantifying cardiac function. The system can include memory that stores strain data representing strain for each of a plurality of myocardial segments of a chamber of a patient's heart. The strain data includes an indication of peak strain and an end-systolic strain for each of the plurality of myocardial segments. A strain delay index calculator is programmed to compute a strain delay index for the patient's heart as a summation of a difference between the peak strain and the end-systolic strain for each of the plurality of myocardial segments.

### BRIEF DESCRIPTION OF THE DRAWINGS

FIG. 1 is a graph depicting strain as a function of time for a post-systolic segment.

FIG. 2 is a graph depicting strain as a function of time for a pre-systolic segment.

FIG. 3 is a flow diagram of a method for characterizing cardiac function.

FIG. 4 depicts a functional block diagram of a system that can be utilized for computing strain delay index.

FIG. 5 is a sample image that can be used for determining strain of myocardial segments.

FIG. 6 is a diagrammatic representation of myocardial segments that can be analyzed for determining strain.

FIG. 7 depicts strain curves for a plurality of myocardial segments as well as a global strain curve.

FIG. 8 depicts an image of an image of a heart chamber before CRT illustrating a plurality of segments that can be used for determining strain thereof.

FIG. 9 depicts a graph depicting strain characteristics as a function of time for the plurality of segments of FIG. 8.

FIG. 10 depicts an image of an image of a heart chamber after CRT illustrating a plurality of segments that can be used for determining strain thereof.

FIG. 11 depicts a graph depicting strain characteristics as a function of time for the plurality of segments of FIG. 10.

FIG. 12 depicts strain curves computed for significantly desynchronized segments.

FIG. 13 depicts strain curves computed for desynchronized segments having different amounts of residual contractility.

FIG. 14 is an example computing environment that can be utilized to perform methods according to an aspect of the invention.

### DETAILED DESCRIPTION

The invention relates to systems and methods to characterize cardiac function. The approach described herein characterizes cardiac function by determining a component of wasted contraction, which is referred to herein as a strain delay index. The strain delay index can be contrasted to an approach that simply quantifies left ventricular (LV) dyssynchrony. In desynchronized myocardium, for example, contractility in delayed segments does not fully contribute to LV end-systolic (ES) function. The strain delay index enables one to quantify an amount of wasted contraction by such delayed segments. This component of wasted contraction (represented by the strain delay index) thus may be utilized as part of cardiac resynchronization therapy (CRT), for example, to improve global ventricular performance, reduce LV wall stress and mitral regurgitation and ultimately lead to reverse remodeling. The strain delay index can also be utilized for predicting response to CRT.

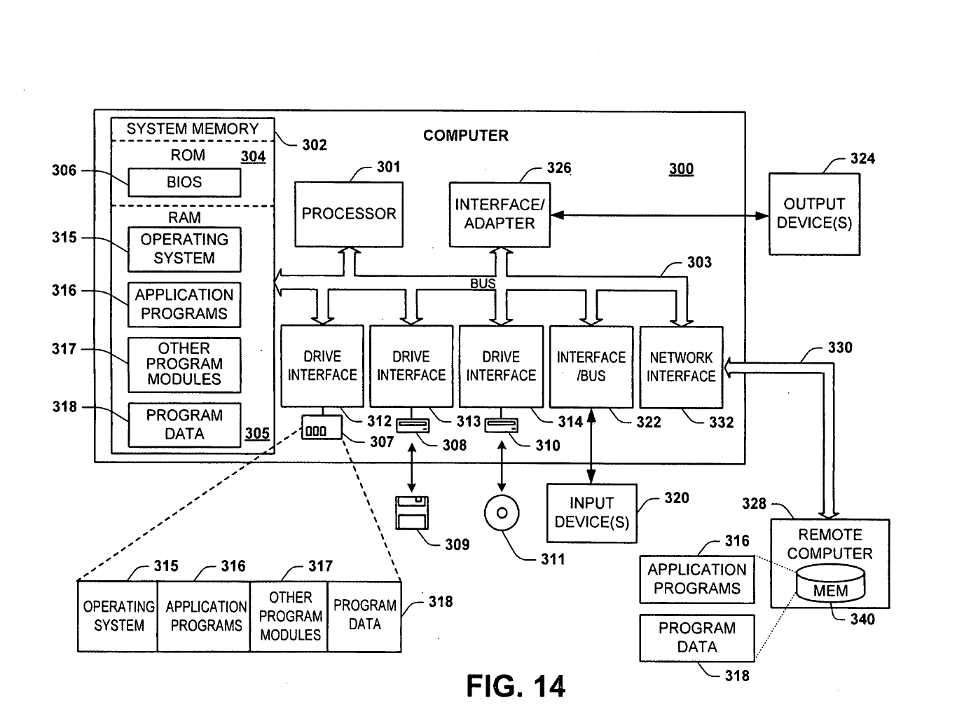

Those skilled in the art will appreciate that portions of the invention may be embodied as a method, data processing system, or computer program product. Accordingly, these portions of the present invention may take the form of an entirely hardware embodiment, an entirely software embodiment, or an embodiment combining software and hardware, such as shown and described with respect to the computer system of FIG. 14. Furthermore, portions of the invention may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer (e.g., an imaging workstation), or other programmable data processing apparatus (or a combination of devices and circuits) to produce a machine, such that the instructions, which execute via the processor, implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

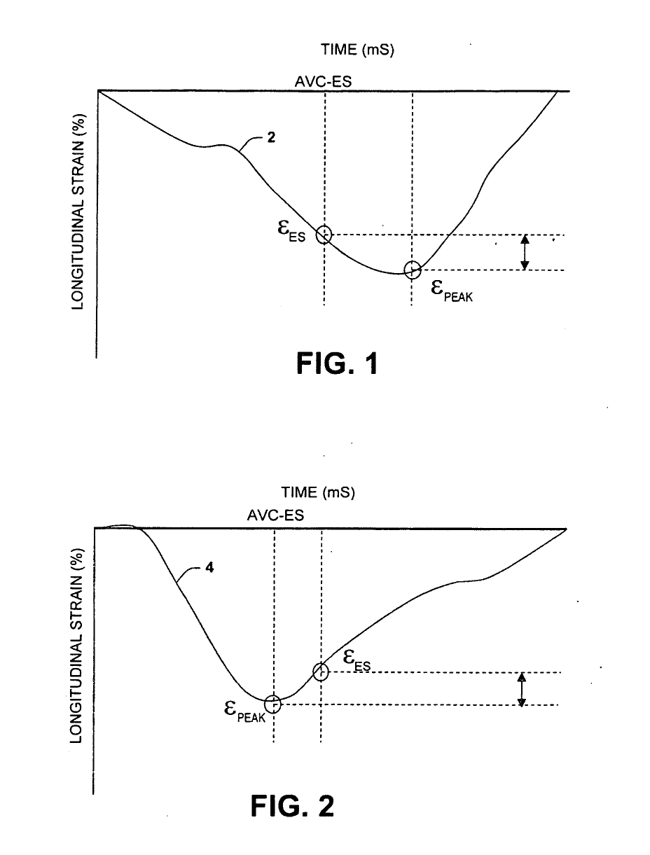

The peak strain of a segment of dyssynchronized myocardium does not fully contribute to end-systolic function. FIGS. 1 and 2 depict example strain curves for two different myocardial segments that exhibit dyssynchrony. FIG. 1 depicts a strain curve 2 for a post-systolic segment in which the peak strain (εₚₑₐₖ) is delayed relative to the aortic valve closure (AVC) at end systole (ES). The wasted energy for such post-systolic segment can be characterized as the difference between the peak strain (εₚₑₐₖ) and the strain at ES (ε_{ES}). FIG. 2 depicts a strain curve 4 for a pre-systolic segment in which the wasted energy can be characterized as the difference between the peak strain (εₚₑₐₖ) and the strain at ES (ε_{ES}).

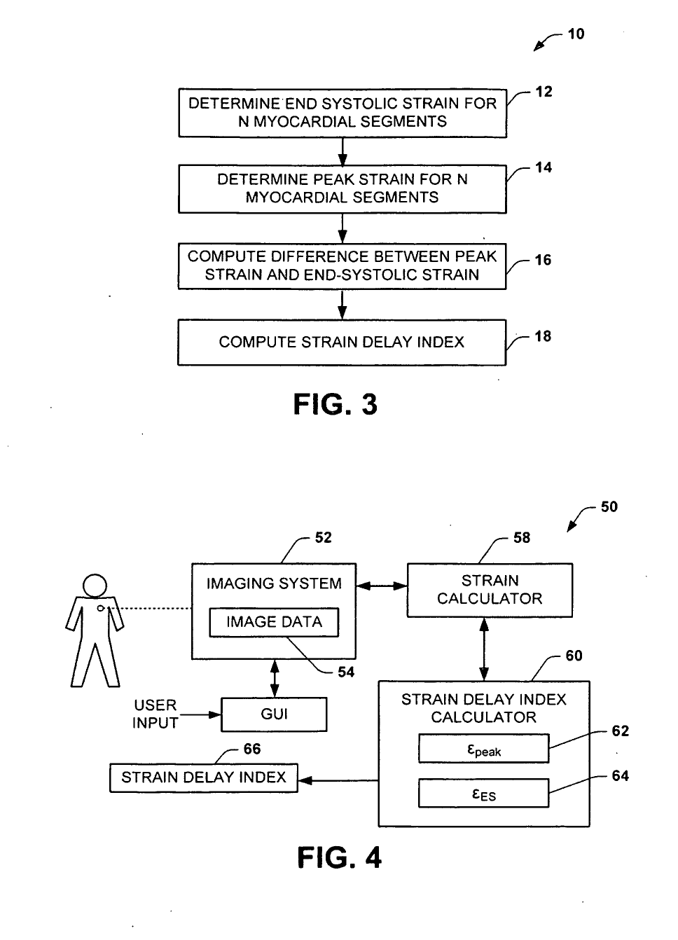

FIG. 3 is a flow diagram depicting a method 10 to quantify cardiac function by determining a component of wasted contraction, namely, a strain delay index. The method 10 operates based on strain data for a plurality of regions of interest, which are referred to herein as myocardial segments. As used herein, strain of a myocardial segment is a geometrical measure of deformation representing the relative displacement of the segment of tissue. Strain thus provides a metric as to the amount of stretch or compression for myocardial tissue segments.

Various models have been developed to divide or segment anatomical regions of the heart into defined myocardial segments. Such models divide the left ventricle into different subdivisions according to image cross-sections taken along different axes thereof. As one example, the ventricle can be divided into the following sixteen segments: septal basal (SB), lateral basal (LB), inferior basal (IB), anterior basal (AB), posterior basal (PB), anterior septal basal (ASB), septal midpapillary (SM), lateral midpapillary (LM), inferior midpapillary (IM), anterior midpapillary (AM), posterior midpapillary (PM), anterior septal midpapillary (ASM), septal apical (SA), lateral apical (LA), inferior apical (IA), and anterior apical (AA). Those skilled in the art will understand that there can be other numbers of myocardial segments, which may be fewer or greater than the sixteen listed above. For instance, twelve (or more) segments can also be utilized.

Additionally, strain curves for a plurality of segments can be determined based on the quantified regional wall motion. Those skilled in the art will appreciate that several methods exist, including but not limited to those described herein, which can be employed to quantify regional wall motion and used to determine strain characteristics for myocardial segments. For instance, imaging systems can be programmed to compute strain and generate corresponding strain curves. Alternatively, imaging data can be acquired for the patient's heart and subsequently analyzed to compute the strain and generate strain curves. The systems and methods described herein are not intended to be limited to any particular imaging modality and may be implemented using various types of two-dimensional and three-dimensional imaging modalities. The strain curves can be generated based on image data in the form of a plurality of sequential frames, such as from one or more cardiac cycle. The method 10 can utilize strain curves computed for all or for a subset of identifiable myocardial segments.

At 12, an end-systolic strain is determined for a plurality of N myocardial segments, where N is a positive integer denoting the number of segments utilized in the method 10. The end-systolic strain for a given segment corresponds to the strain (e.g., on a strain curve) at a time that coincides with end systole. As an example, end systole can correspond to aortic valve closure. This can be determined visually from the image data. Alternatively, end systole can be determined from an electrocardiogram (EKG) that can be recorded and synchronized with the image data. Those skilled in the art will understand and appreciate various ways to determine end systole, any of which can be utilized for performing the method 10.

At 14, peak strain for each of the N myocardial segments is determined. The peak strain can be ascertained from strain curves by identifying a maximum strain value. At 16, the difference between the peak strain (from 14) and the end-systolic strain (from 12) is computed for each of the N myocardial segments. This difference quantifies an amount of wasted contraction for each respective segment.

A strain delay index value is computed at 18 as a function of the peak strain and the end-systolic strain across the N myocardial segments. The strain delay index can be expressed mathematically as equal to the sum of the difference between peak (εₚₑₐₖ) and end-systolic strain (ε_{ES}) across the (n) myocardial segments, which can be represented as follows:

$$Strain delay Index = {\sum }_{i = 1}^{n} ( {{ɛ}_{peak}}_{i} - {ɛ}_{{ES}_{i}} ) \qquad Eq . 1$$

The strain delay index computed at 18 expresses a difference of contractility amplitude. The strain delay index can be normalized according the number of segments. The differences (εₚₑₐₖ−ε_{ES}) for each of the myocardial segments can also be aggregated or otherwise be analyzed by other mathematical and statistical methods.

FIG. 4 depicts a functional block diagram of a system 50 programmed and configured to compute strain delay index according to an aspect of the invention. The system 50 includes an imaging system 52 that acquires image data for a patient's heart over one or more cardiac cycles. Those skilled in the art will understand that various types of imaging modalities can be utilized to quantify regional wall motion, although the accuracy of the computations generally depends on the precision of the method for quantifying regional wall motion.

For example, the imaging system 52 can be implemented as including an ultrasound imaging device and associated workstation programmed to perform two-dimensional speckle tracking, which is an echocardiographic modality that enables angle-independent assessment of myocardial deformation indices. Other types of cardiac imaging modalities that could be utilized as the imaging system 52 include electrocardiography, radiography, computed tomography (CT), magnetic resonance imaging (MRI), echocardiography, nuclear imaging and positron emission tomography (PET). While the approach described herein is explained in the context of two-dimensional image data, the concept is applicable to and may be extended to three-dimensional imaging techniques. It will be understood that the image data is acquired with respect to time and thus, having a time component, the two-dimensional imaging can be considered three-dimensional (e.g., having two geometrical axes and one time axis). Similarly, the three-dimensional imaging mentioned would also be acquired for a plurality of frame with respect to time, which can be considered four-dimensional (e.g., having three geometrical axes and one time axis).

The imaging system 52 thus provides image data 14, such as including data that represents a plurality of segments of the cardiac wall during the at least a portion of a cardiac cycle. For instance, the image data can be from a single cardiac cycle or image data from a plurality of cycles can be aggregated, such that the strain curves are produced for each segment based on the average strain computed over a plurality of cardiac cycles. The image data can includes markers or other identifying information that can be tracked for each of a plurality n of myocardial segments, where n is a positive integer denoting the number of tissue segments. Each segment defines a region interest of myocardial tissue, such as described herein.

As one example, the image data 54 can be acquired via ultrasound employing two-dimensional (2-D) speckle tracking. Because of scattering, reflection and interference of the ultrasound beam in myocardial tissue, speckles appear in grey scale 2-D echocardiographic images. These speckles represent tissue markers that can be tracked from frame to frame throughout the cardiac cycle. Each speckle can be identified and tracked, corresponding to a myocardial segment, by calculating frame to frame changes—similar to analysis with tagged cMR—using a sum of absolute difference algorithms. Motion can also be analyzed for the myocardial segments by integrating frame to frame changes.

Commercially available or proprietary software can be implemented as part of the image system 52 to perform the spatial and temporal processing of these speckles acquired from the 2-D echocardiograph images. For example, the Vivid™ 7 Dimension system and the EchoPAC™ Dimension workstation, both available from the GE Healthcare division of the General Electric Company, can be utilized as the image system 52 to acquire and generate the image data 54. Such systems also may be programmed to generate strain curves for the myocardial segments.

These and other commercially available products may include a variety of mechanisms for defining the plurality of segments in the image data, which may be manual, semi-automated or fully automated processes. The particular approach can vary according to the type of imaging system 52 and available methods. As one example, the user can employ a graphical user interface (GUI) 56 to trace or outline the internal border of the myocardium. The border can be parallel to anatomical direction of the longitudinal contraction and relaxation. Alternatively, the segments can be identified semi-automatically or automatically. For a semi-automatic approach, the user can employ the GUI 56 mark a plurality of points on the image of the heart, such as at the annulus and at the apex. The imaging system 52 can employ computer-implemented methods to assess the placement of the points and construct boundaries for the segments. If the points may be misplaced, the imaging system 52 can be programmed to identify instances where the points have been misplaced and correct the position of the points.

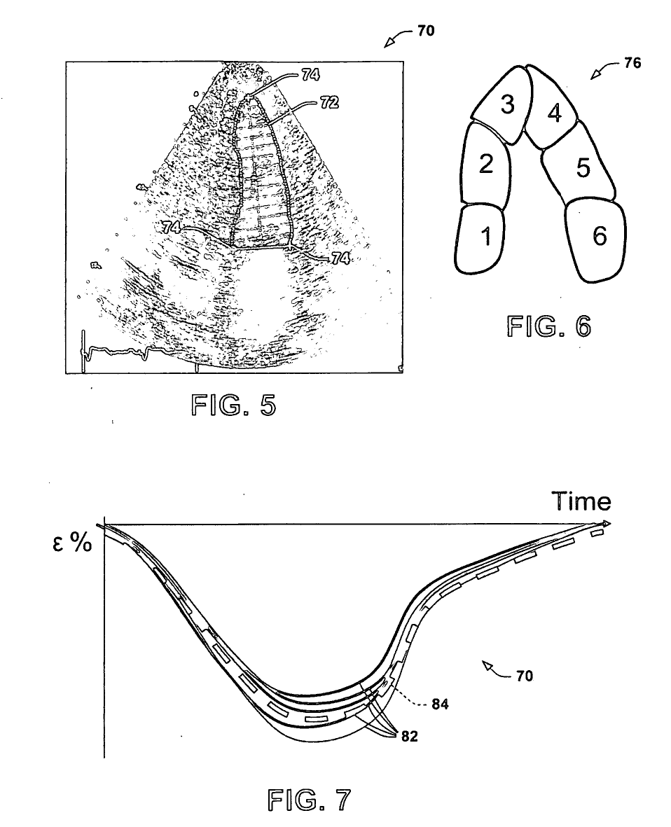

FIG. 5 depicts an example of an ultrasound speckle tracking image 70 of a patient's left ventricle at an instance in time of the cardiac cycle. In this example image 70 an inner boundary 72 of the myocardium is superimposed on the image parallel to the direction of longitudinal contraction. For instance, software of the imaging system 52 can generate the boundary 72 based on points 74 marked by the user.

FIG. 6 depicts an example of six segments 76 which can correspond to regions of interest for the myocardial tissue shown in the image of FIG. 5. Those skilled in the art will understand several ways in which the segments can be represented in an image and analyzed.

Returning to FIG. 4, the system 50 also includes a strain calculator 58 that is programmed to compute strain values for each of plurality of myocardial segments throughout the cardiac cycle. The strain calculator 58 analyzes boundaries for each of the segments in the image data and generates strain curves for each such segment (or data from which strain curves can be generated) based on the image data 54. As described herein, the image data can correspond to multiple sets of images taken along different axes of one or more heart chamber.

There are various ways that the strain calculator can be implemented, including manual or automatic methods. For instance, the strain calculator 58 can be implemented as a software product that can be executed on a machine separately from the imaging system 52 to compute strain curves for the myocardial segments based on the image data 54 acquired by the imaging system. Alternatively, the strain calculator 58 can be implemented as part of the imaging system 52, as can be found in many commercially available imaging system, such as mentioned herein. The strain calculator 58 can provide the computed strain as an output, which can be visualized (e.g., on a display or printer), such as in the form of a strain curve for each of the plurality of myocardial segments.

The strain calculator 58 can also compute a global strain curve, such as can be defined as the mean (or average) regional strain value with respect to time. For instance, the global strain curve can be derived to represent the whole LV function, such by averaging the regional LV strain curves incrementally along (e.g., at every 2.5% of) the cardiac cycle for the plurality of myocardial segments. The time to peak point of the global strain curve can be used to define the timing of ES, although other methods can also be used to define the ES timing.

FIG. 7 depicts an example graph 80 illustrating sample strain curves 82, such as can be generated for a plurality of myocardial segments. Also shown in FIG. 7 is an example of a corresponding global strain curve 84, such as can be computed by the strain calculator 58 by averaging the strain curves.

The system 50 also includes a strain delay index calculator 60 that is programmed to compute a strain delay index 66 according to an aspect of the invention. The program instructions can reside in memory as part of a computer that may be part of the imaging system 52. The imaging system, for instance, can be programmed to compute the strain delay index 66, such as in response to a user input to GUI 56. Alternatively, the instructions can run on a computer or workstation that is separate from the imaging system 52 and to which the image data 54 (or a selected subset thereof) and/or strain data are loaded. For example, the computations performed by the strain delay index calculator 60 can be performed automatically in any appropriate mathematical tool, such as Excel® available from Microsoft Corporation of Redmond, Wash., that is programmed to perform such analysis. As yet another alternative, the strain delay index calculator 60 function can be performed manually, such as based on the strain curves produced by the strain calculator 58.

The strain delay index calculator 60 can determine a value for the peak contractility (or peak strain), indicated at 62, for each of the plurality of myocardial segments. The strain delay index calculator 60 can also determine the timing of the end of systole (ES). As described herein, the ES timing value can be determined as the time value at which the global strain curve peaks. This ES timing value can provide an index to the strain curves and used to determine a value for the end-systolic contractility (or ES strain), indicated at 64, for each of the plurality of myocardial segments.

The strain delay index calculator 60 in turn computes the strain delay index 66 as the summation of a difference between peak contractility and end-systolic contractility across the plurality of myocardial segments, such as expressed mathematically in Eq. 1. Strain delay index has been determined to be correlated with reverse remodeling in both ischemic and non ischemic patients. For instance, it has been determined from receiver operating characteristic curves for diagnosis of response to CRT that a strain delay index value of approximately 25% or greater can be utilized to identify responders with about 90% positive and negative predictive value. Advantageously, the strain delay index has better predictive value than many other known predictive metrics, including SD-TDI for response to CRT, in both ischemic and non-ischemic patients.

In view of the foregoing, systems and methods that can be implemented in accordance with the invention will be better appreciated in view of the discussion with respect to FIGS. 8-11.

FIG. 8 depicts an example ultrasound speckle tracking image 100 of a patient's heart, including the left ventricle at end systole (ES). The image 100 shows the ventricle before performing CRT. For instance, a heart exhibiting ventricular dyssynchrony can have an ES volume (ESV) of about 113 ml, generally corresponding to the volume of blood remaining in the heart at ES. Also depicted in the image 100, is a representation 102 for defining the inner myocardial boundary of the left ventricle. Such a boundary 102 can be generated by marking the image via a GUI of an imaging workstation, for example. Disposed in a substantially spaced apart relationship along the boundary 102 are a plurality of myocardial segments, as indicated by circular graphical elements 104, 106, 108, 110, 112, and 114.

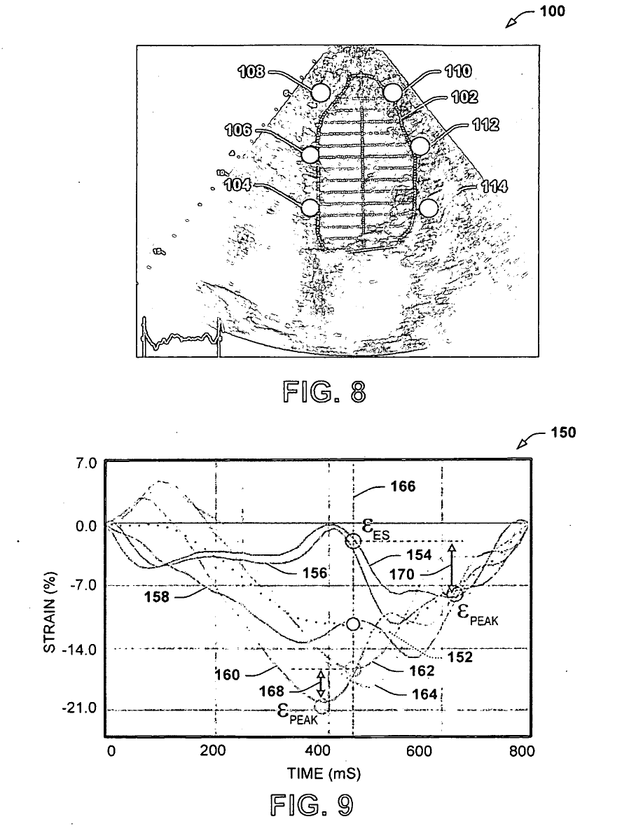

FIG. 9 is a graph 150 illustrating a plurality of strain curves 152, 154, 156, 158, 160, 162 and 164. The strain curve 152 (illustrated as a dotted line) corresponds to the global strain (e.g., average strain) for the set of myocardial segments. The other strain curves 154, 156, 158, 160, 162 and 164 depict the strain computed for each of the myocardial segments 104, 106, 108, 110, 112, and 114 shown in FIG. 8, respectively. The timing for end systole, demonstrated at 166, thus can correspond to the peak of the global strain curve 152.

As a further illustration, wasted energy associated with strain curve 160 (corresponding to segment 110) is shown at 168, which corresponds to the difference between the peak strain ε_{PEAK} and the ES strain ε_{ES} for the curve 160. Wasted energy associated with strain curve 154 (corresponding to segment 104) is shown at 170, which corresponds to the difference between the peak strain ε_{PEAK} and the ES strain ε_{ES} for curve 154. Similar differences between peak and ES strain can be computed for each of the other curves, which can be summed together to provide a corresponding strain delay index value such as described herein.

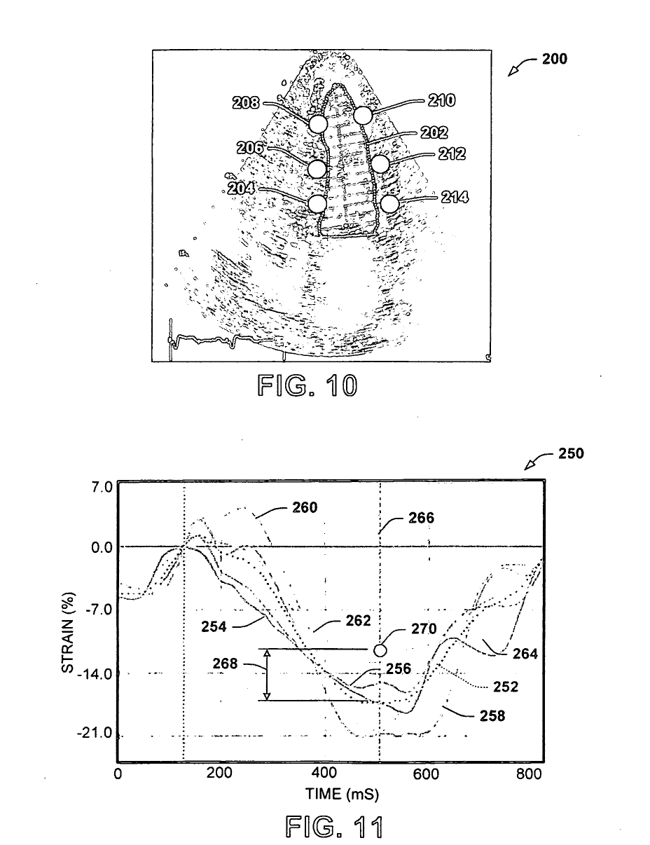

FIG. 10 depicts an example ultrasound speckle tracking image 200 of the same patient's heart as in FIG. 8, demonstrating the left ventricle at end systole (ES). The image 200 shows the same ventricle along the same axis after performing CRT for a period of months (e.g., about three months). Also depicted in the image 200, is a boundary representation 202 for the inner myocardial surface of the left ventricle. Disposed in a substantially spaced apart relationship along the boundary 202 are a plurality of myocardial segments, as indicated by circular graphical elements 204, 206, 208, 210, 212, and 214. The segments are substantially the same as in the example of FIG. 8, although after CRT.

FIG. 11 is a graph 250 illustrating a plurality of strain curves 252, 254, 256, 258, 260, 262, and 264. As in the example of FIG. 10, the strain curve 152 (illustrated as a dotted line) corresponds to the global strain (e.g., average strain) for the set of myocardial segments. The other strain curves 254, 256, 258, 260, 262, and 264 depict the post-CRT strain computed for each of the myocardial segments 204, 206, 208, 210, 212, and 214 shown in FIG. 10, respectively. The timing for end systole is also shown in FIG. 11 at 266.

A comparison of FIG. 11 and FIG. 9 demonstrates a significant re-synchronization of the myocardial segments after CRT. For example, the ESV for the left ventricle before CRT was 113 ml, whereas after CRT the ventricle was determined to have an ESV of 50 ml. It will be appreciated that the overall increase in contractility resulting from reverse remodeling due to CRT can be predicted based on computing the strain delay index for the pre-CRT data of FIG. 9, as shown and described herein. The increase in the global strain curve 252 is indicated at 268 as the difference between the strain from FIG. 9 (indicated at 270) and the peak global strain. The increase in global strain curve is expected to be proportional to the strain delay index.

Those skilled in the art will understand and appreciate various ways to graphically represent the strain delay index and the amount of wasted contraction (εₚₑₐₖ−ε_{ES}) computed for each of the plurality of segments. Additionally or alternatively, the strain delay index can be compared to a predefined threshold (or thresholds) to ascertain an objective indication of the dyssynchrony. For instance, one or more thresholds can be defined statistically based on clinical studies that relate the strain delay index relative to known amounts of dyssynchrony. Additionally, the strain delay index can be combined with one or more other predictors (e.g., velocity data acquired by tissue Doppler imaging (TDI), interrogating myocardial viability, and contractile reserve) to identify and predict responders to CRT.

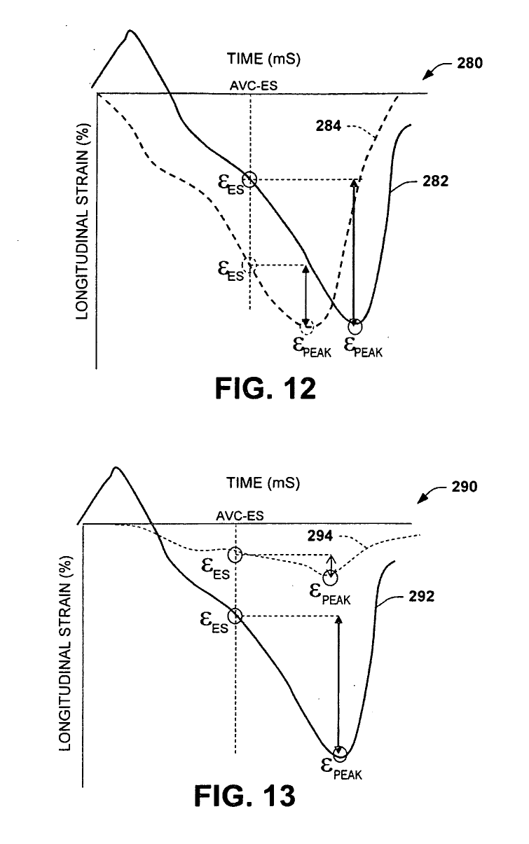

By way of further example, delayed segments incrementally impact the strain delay index value not only in proportion to the severity of dyssynchrony but also relative to the amplitude of their residual contractility. This is because the difference (ε_{PEAK}-ε_{ES}) is low (e.g., about ≦1%) in non desynchronized (<5% delay from end systole) or severely dysfunctional segments (ε_{PEAK}<−5%). For instance, FIG. 12 is a graph 280 depicting strain curves 282 and 284 for segments exhibiting different amounts of dyssynchrony and comparable peak strain ε_{PEAK}. Thus the difference (ε_{PEAK}−ε_{ES}) for each segment varies according to the amount of dyssynchrony. It is thus expected that the wasted energy due to dyssynchrony in each segment increases with the severity of the delayed contraction. By way of further comparison FIG. 13 is graph 290 of strain curves 292 and 294. The curve 294 represents strain for a scarred myocardial segment. In FIG. 14, each of the curves 292 and 294 have comparable dyssynchrony, although contrasted differences (ε_{PEAK}−ε_{ES}). From FIG. 13 it is demonstrated that a scarred segment whose contractility has little likelihood to improve with resynchronization therapy will barely increase the strain delay index despite the presence of significantly delayed contraction since its εpeak and εES differ only slightly. The difference (ε_{PEAK}−ε_{ES}) would be greater in a myocardial segment with preserved contractility (e.g., represented by strain curve 292) than in those with no or minimal residual contractility, as in scar or fibrotic myocardial tissue (e.g., represented by curve 294).

It will be understood that systems and methods implemented according to the present invention can predict response to CRT based on the assessment of a component of impaired contractility related to dyssynchrony which can be inferred as the acute gain of contractility expected after resynchronization. The acute increase in myocardial performance plays an important role for the long term effects of CRT since it will help to reduce LV wall stress and mitral regurgitation and trigger the reverse remodeling process. The degree of impaired contractility expressed by the strain delay index was not only derived from delayed segments but also from pre-systolic segments. Time to peak strain in pre-systolic segments are not expected to change with CRT but the recruitment of delayed segments in addition to an earlier occurrence of the end-systolic events enable pre-systolic segments to fully contribute to myocardial function.

As mentioned above, the strain delay index is expected to have similar accuracy in patients with ischemic and non ischemic cardiomyopathies. Such accuracy can result where a greater number of myocardial segments (e.g., sixteen segments) of the ventricle are utilized to compute the strain delay index. Such an index is further more robust than existing methods since the strain delay index is not a simple measurement of contractility or time delay but a combination (and relative weighting) of both of these parameters.

In view of the foregoing, FIG. 14 illustrates one example of a computer system 300 that can be employed to execute one or more embodiments of the invention by storing and/or executing computer executable instructions. Computer system 300 can be implemented on one or more general purpose networked computer systems, embedded computer systems, routers, switches, server devices, client devices, various intermediate devices/nodes or stand alone computer systems. Additionally, computer system 300 can be implemented on various mobile clients such as, for example, a personal digital assistant (PDA), laptop computer, pager, and the like, provided it includes sufficient processing capabilities.

Computer system 300 includes processing unit 301, system memory 302, and system bus 303 that couples various system components, including the system memory, to processing unit 301. Dual microprocessors and other multi-processor architectures also can be used as processing unit 301. System bus 303 may be any of several types of bus structure including a memory bus or memory controller, a peripheral bus, and a local bus using any of a variety of bus architectures. System memory 302 includes read only memory (ROM) 304 and random access memory (RAM) 305. A basic input/output system (BIOS) 306 can reside in ROM 304 containing the basic routines that help to transfer information among elements within computer system 300.

Computer system 300 can include a hard disk drive 307, magnetic disk drive 308, e.g., to read from or write to removable disk 309, and an optical disk drive 310, e.g., for reading CD-ROM disk 311 or to read from or write to other optical media. Hard disk drive 307, magnetic disk drive 308, and optical disk drive 310 are connected to system bus 303 by a hard disk drive interface 312, a magnetic disk drive interface 313, and an optical drive interface 314, respectively. The drives and their associated computer-readable media provide nonvolatile storage of data, data structures, and computer-executable instructions for computer system 300. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of the present invention.

A number of program modules may be stored in drives and RAM 305, including operating system 315, one or more application programs 316, other program modules 317, and program data 318. The application programs 316 and program data 318 can include functions and methods programmed to determine a strain delay index as well as to perform other related computations or associated functionality, such as described herein.

A user may enter commands and information into computer system 300 through one or more input devices 320, such as a pointing device (e.g., a mouse, touch screen), keyboard, microphone, joystick, game pad, scanner, and the like. For instance, the user can employ input device 320 to edit or modify a domain model. Additionally or alternatively, a user can access a user interface via the input device to create one or more instances of a given domain model and associated data management tools, as described herein. These and other input devices 320 are often connected to processing unit 301 through a corresponding port interface 322 that is coupled to the system bus, but may be connected by other interfaces, such as a parallel port, serial port, or universal serial bus (USB). One or more output devices 324 (e.g., display, a monitor, printer, projector, or other type of displaying device) is also connected to system bus 303 via interface 326, such as a video adapter.

Computer system 300 may operate in a networked environment using logical connections to one or more remote computers, such as remote computer 328. Remote computer 328 may be a workstation, computer system, router, peer device, or other common network node, and typically includes many or all the elements described relative to computer system 300. The logical connections, schematically indicated at 330, can include a local area network (LAN) and a wide area network (WAN).

When used in a LAN networking environment, computer system 300 can be connected to the local network through a network interface or adapter 332. When used in a WAN networking environment, computer system 300 can include a modem, or can be connected to a communications server on the LAN. The modem, which may be internal or external, can be connected to system bus 303 via an appropriate port interface. In a networked environment, application programs 316 or program data 318 depicted relative to computer system 300, or portions thereof, may be stored in a remote memory storage device 340.

What have been described above are examples and embodiments of the invention. It is, of course, not possible to describe every conceivable combination of components or methodologies for purposes of describing the invention, but one of ordinary skill in the art will recognize that many further combinations and permutations of the present invention are possible. Accordingly, the invention is intended to embrace all such alterations, modifications and variations that fall within the scope of this application, including the appended claims.

What is claimed is:

1. A method for quantifying cardiac function for a patient's heart, comprising:
determining an end-systolic strain for each of a plurality of myocardial segments;
determining a peak strain in each of the plurality of myocardial segments;
computing a difference between the peak strain and the end-systolic strain for each of the plurality of myocardial segments; and
computing a strain delay index from the computed differences.

2. The method of claim 1, generating strain curves for each of the plurality of myocardial segments, the end-systolic strain and the peak strain for each of the plurality of myocardial segments being ascertained from the respective strain curves.

3. The method of claim 2, further comprising determining a global strain curve by averaging the strain curves with respect to time, the global strain curve representing overall strain for ventricular function.

4. The method of claim 2, further comprising determining a timing of end systole as a time at which the global strain curve peaks.

5. The method of claim 1 further comprising:
quantifying regional wall motion for a ventricle of the patient' heart; and
determining longitudinal strain for the plurality of myocardial segments of the ventricle based on the quantified regional wall motion.

6. The method of claim 5, wherein the quantifying regional wall motion further comprises acquiring images of the patient's heart over time to provide corresponding image data, the corresponding image data including a representation of wall motion for the plurality of myocardial segments; and
processing the corresponding image data to provide strain curves for the plurality of myocardial segments, the end-systolic strain and the peak strain for each of the plurality of myocardial segments being ascertained from the respective strain curves.

7. The method of claim 6, wherein the acquiring images further comprises employing an ultrasound imaging modality.

8. The method of claim 7, wherein the ultrasound imaging modality comprises two-dimensional speckle tracking echocardiography.

9. The method of claim 7, wherein the acquiring images further comprises employing one of a computed tomography imaging modality and a magnetic resonance imaging modality.

10. The method of claim 6, further comprising:
determining a global strain curve by averaging the strain curves with respect to time, the global strain curve representing overall strain for the ventricle; and
determining timing of end systole as the time at which the global strain curve peaks.

11. The method of claim 5, wherein the plurality of myocardial segments comprise at least twelve myocardial segments.

12. The method of claim 5, wherein the longitudinal strain for the plurality of myocardial segments comprises strain longitudinal strain for at least sixteen myocardial segments of the ventricle.

13. The method of claim 1, wherein the strain delay index is defined as follows:
$$strain delay index = {\sum }_{i = 1}^{n} ( {{ɛ}_{peak}}_{i} - {ɛ}_{{ES}_{i}} )$$
where:
εₚₑₐₖ is the peak strain for a given segment i of the plurality of myocardial segments;
ε_{ES} is the end-systolic strain for the given segment i; and
n denotes a number of plurality of myocardial segments.

14. A method for quantifying cardiac function for a patient's heart comprises computing a summation of a difference between peak contractility and end-systolic contractility across a plurality of myocardial segments of a chamber of the patient's heart to provide a strain delay index, whereby a response to cardiac resynchronization therapy is predictable according to a value of the strain delay index.

15. The method of claim 14, further comprising generating strain curves for each of the plurality of myocardial segments from which peak strain and end-systolic strain are determined for each of the plurality of myocardial segments, the difference between peak contractility and end-systolic contractility being ascertained from the strain curves for the respective plurality of myocardial segments.

16. A system for quantifying cardiac function, comprising:
memory that stores strain data representing strain for each of a plurality of myocardial segments of a chamber of a patient's heart, the strain data including an indication of peak strain and an end-systolic strain for each of the plurality of myocardial segments; and
a strain delay index calculator that is programmed to compute a strain delay index for the patient's heart as a summation of a difference between the peak strain and the end-systolic strain for each of the plurality of myocardial segments.

17. The system of claim 16, further comprising means for determining timing for end systole.

18. The system of claim 16, further comprising an imaging system that acquires images of the chamber of the patient's heart and stores corresponding image data in the memory, the corresponding image data including a representation of wall motion for the plurality of myocardial segments,
wherein one of the imaging system or the strain delay index calculator is programmed to process the corresponding image data to generate strain curves for the plurality of myocardial segments, the end-systolic strain and the peak strain being ascertained from the respective strain curves.

19. The system of claim 18, wherein the imaging system further comprises two-dimensional speckle tracking echocardiography.

20. The system of claim 18, wherein the imaging system further comprises one of a computed tomography imaging modality and a magnetic resonance imaging modality.

21. The system of claim 16, wherein the plurality of myocardial segments comprises at least sixteen myocardial segments of the ventricle of the patient's heart.
